(19) United States
(12) Patent Application Publication
(10) Pub. No.: US 2017/0231523 A1
(43) Pub. Date: Aug. 17, 2017

(54) METHOD AND MAGNETIC RESONANCE APPARATUS SCAR QUANTIFICATION IN THE MYOCARDIUM

(71) Applicant: Siemens Healthcare GmbH, Erlangen (DE)

(72) Inventor: Andreas Greiser, Erlangen (DE)

(73) Assignee: Siemens Healthcare GmbH, Erlangen (DE)

(21) Appl. No.: 15/429,299

(22) Filed: Feb. 10, 2017

(30) Foreign Application Priority Data
Feb. 11, 2016 (DE) 102016202085.4

(51) Int. Cl. A61B 5/055; A61B 5/00; G01R 33/563; G01R 33/56; G01R 33/50; G01R 33/54

(52) U.S. Cl. CPC A61B 5/055; G01R 33/50; G01R 33/543; G01R 33/563; G01R 33/5601; A61B 5/7246; A61B 5/0044; A61B 2576/023

(57) ABSTRACT

In a method and magnetic resonance (MR) apparatus for determining a fraction of scar tissue in the myocardium of an examination person, magnetization of nuclear spins is prepared by radiation of a preparation pulse in the myocardium, and MR signals are acquired for multiple MR images while the magnetization returns to equilibrium. The multiple MR images are brought into registration with each other, so a movement of the heart between MR images is compensated. T1 times are determined using this sequence of compensated MR images. Different MR template images with different contrasts are calculated at different times after radiation of the preparation pulse, using the calculated T1 times. A myocardial contour is determined using one of the template images that has a first contrast. Scar tissue in the myocardium is determined using another template image that has a second contrast that differs from the first contrast.

### BACKGROUND OF THE INVENTION

Field of the Invention

The present invention concerns a method for determining a fraction of scar tissue in the myocardium of an examination person, and to a magnetic resonance (MR) system for implementing such a method.

The invention also concerns an electronically readable data carrier encoded with programming instructions that cause such a method to be implemented.

Description of the Prior Art

An established MR imaging method for determining the vitality of a myocardium is imaging known as “Late Gadolinium Enhancement”, LGE imaging. Here, contrast medium is administered intravenously to an examination person and after several minutes images of the heart are recorded, with an inversion pulse being radiated for signal preparation, with the interval for signal readout being chosen such that healthy myocardium is exactly at the zero crossing of the signal. This means that the healthy myocardium does not make a signal contribution in the associated MR image. Scarred tissue in the myocardium has stored more contrast medium therein, so the T1 time thereof is reduced and the scar tissue appears bright.

A clinically valuable parameter is quantification of the scar tissue, i.e. determining the volume fraction of the scarred tissue in the myocardium relative to the total volume of the myocardium. Segmenting the entire myocardium onto the LGE images is difficult, however, since the contrast between myocardium and blood and the surrounding lung tissue is slight, and this makes accurate segmenting difficult. To solve this problem, other image data have been used in conventional approaches, wherein, for example, the cardiac phase corresponding to LGE-data can be chosen from TrueFISP-image series from the same slice. Nevertheless, there remains the problem of respiratory movement since the independent data are recorded in different breath-holding phases, and therefore often has a considerable offset. This leads to a manual, time-consuming correction of the contouring, and the problem thus occurs that the LGE data themselves do not actually have the necessary contrast.

### SUMMARY OF THE INVENTION

An object of the present invention is to further improve determination of the fraction of scar tissue in the myocardium.

According to a first aspect of the invention, a method for determining the fraction of scar tissue in the myocardium of an examination person is provided wherein magnetization of nuclear spins is prepared by radiation of at least one preparation pulse into an examination region that includes the myocardium. Furthermore, MR signals are recorded from the examination region while the magnetization approaches a state of equilibrium (returns to the steady state). The acquired MR signals are used to reconstruct MR images that are brought into registration with each other, so a movement of the heart between the multiple MR images is compensated with different contrasts, so a sequence of movement-compensated MR images results. T1 times are determined in the examination region with the use of the sequence of compensated MR images. Using the calculated T1 times, different MR template images of the examination region can be calculated at different times after radiation of the at least one preparation pulse, with the different MR template images having different contrasts in the examination region. A myocardial contour is determined with the use of a myocardium template image that has a first contrast and was chosen from the MR template images. Furthermore, the scar tissue in the myocardium is determined with the use of at least one scar template image that has a second contrast that differs from the first contrast and was chosen from the MR template images.

Different MR images, the MR template images that have different contrasts, can be calculated from the calculated T1 values in the various image points of the examination region. Due to the different contrasts, it is possible to choose a myocardium template image from the MR template images that has the best contrast for determining the myocardial volume and thus is best for segmenting the myocardium. A different template image can be chosen as the scar template image in which the scar in the myocardium has the best contrast in relation to the surrounding myocardium. The basis of the present invention is therefore to calculate T1 times and then the template images with different contrasts for the purpose of segmenting, instead of using the LGE images themselves or also other recorded MR images, so the optimum scar-myocardium contrast can be chosen, and the optimum contrast of the overall myocardium in relation to the surrounding tissue also can be selected.

The steps mentioned above preferably all occur automatically.

The radiation of the next preparation pulse for recording the MR signals can be chosen such that the magnetization has not yet returned to its state of equilibrium, but has a value that is, for example, less than 70 percent of the state of equilibrium magnetization.

Since it is not the absolute quantification of the T1 time that is paramount, but the determination of the myocardial volume and scar volume, the relaxations required for accurate T1 time determination are not very significant, so scanning time can be saved.

Furthermore, the myocardial volume to be determined with the use of two different myocardium template images that have different contrasts, with a myocardium outer limit being determined on a first myocardium template image and a myocardium inner limit being determined on a second myocardium template image. The different fractions of the myocardium thus are not determined in a single MR template image. Instead, a contour or partial contour is determined in a first myocardium template image, and a further myocardial contour or partial myocardial contour is determined in a second myocardium template image.

In an embodiment, the MR signals for generating the multiple MR images are recorded during the same movement phase of the heart, with the MR signals being recorded over a period of at least six heartbeats. In this embodiment, the movement phase of a heart is determined and only MR images recorded, for example with the use of ECG-triggering, when a specific movement phase of the heart is detected. This extends the recording time but the entire recording process is possible within one breath-hold, for example within 10-15 heartbeats.

In a further embodiment, the MR signals for generating the multiple MR images are recorded over different movement phases of the heart, with the MR signals being recorded over a period that encompasses less than five heartbeats. The MR images can be recorded more or less continuously over the different cardiac phases here.

The following steps can be carried out for some of the different movement phases: T1 times are calculated for the respective movement phase using MR images which can be associated with this one movement phase. Furthermore, corresponding MR template images in each case are calculated for the different movement phases at different times after irradiation of the respective preparation pulse. Furthermore, it is possible to choose the MR template images from one of the movement phases and to determine the myocardial volume and the scar volume with the use of the at least one myocardium template image and the at least one scar template image, which were each determined from the MR template images of the chosen movement phase.

Since the myocardium contracts and expands over the different movement phases, for the quantification of the scar tissue fraction it is advantageous to use only template images from one movement phase in each case, otherwise the calculated scar fraction can be incorrect when comparing template images from different cardiac phases. One reason for this is because individual slices are being considered, and a portion of the myocardium moves out of the slice and into the slice due to the cardiac movement. It is possible to calculate the myocardial volume and the scar tissue in the number of cardiac phases here, so an averaged myocardial volume and an averaged scar tissue or an averaged fraction of scar tissue in the myocardium can be determined.

The present invention also encompasses a magnetic resonance system (apparatus) designed to implement the method as described above, wherein an MR control computer is provided to operate the MR scanner of the apparatus so as to radiate the preparation pulse and to record the raw data for the multiple MR images. A processor is provided, furthermore, that is configured to calculate template images and therewith a myocardial volume and scar tissue from the multiple MR images that are brought into registration relative with each other.

The features described above and the features described below can be used not only in the explicitly mentioned combinations, but also in other combinations or in isolation, without departing from the basis of the invention.

### BRIEF DESCRIPTION OF THE DRAWINGS

FIG. 1 is a block diagram of an MR apparatus with which a fraction of scar tissue in the myocardial volume can be inventively determined.

FIG. 2 schematically shows how T1 times and a T1 map are generated from the multiple MR images, with the use of which different MR template images with different contrasts are calculated to then determine a fraction of scar tissue in the myocardial volume.

FIG. 3 schematically shows an image sequence for receiving the MR images, with which the MR template images are then calculated.

FIG. 4 schematically shows how a T1 time can be calculated from intensity values of MR images at different inversion times.

FIG. 5 schematically shows a further embodiment in which MR images are recorded over different cardiac phases, and then a myocardial volume and scar tissue in the myocardium are calculated.

FIG. 6 is a flowchart of steps for implementing a quantification of scar tissue in the myocardium in accordance with the invention.

### DESCRIPTION OF THE PREFERRED EMBODIMENTS

Tissue characterization is an important feature in MRT of the heart since it constitutes a unique feature compared to other imaging methods and provides significant additional information about the physiological state of the cardiac muscle tissue. The quantification of T1 in the myocardium provides information about the tissue state in respect of the integrity of the cell membrane of the cardiac muscle fibers. The further advantage of quantitative MR images with determination of the T1 time is that even slight changes, which are not yet visible on LGE images, can be detected in the tissue.

For generating the T1 maps it is possible to scan a number of images having different inversion times T1 but always in the same cardiac phase, so in principle a T1 relaxation curve can be fitted for each image element. To minimize residual movement effects, the individual MR images can be brought into registration with each other, optionally in an intermediate step for aligning the contrasts. The contrasts in the multiple MR images are very different, with the last images with the highest T1 values having an almost uniform contrast, like imaging sequences without an inversion pulse, in which, inter alia, the myocardium can generally be differentiated very well. With low T1 values (inversion times) the contrast changes significantly in the MR images. Whether the signal zero crossing of the healthy myocardium is accurately met for a T1 value depends on the exact T1 value and on the relevant T1 value. In the present invention, T1 times are accordingly calculated, so any contrast can be calculated for the individual image elements or pixels. An MR image having optimized T1 for the zero crossing of the myocardial signal thus can also be calculated from the T1 values. Optimum blood suppression occurs for a different T1, so segmenting of the entire myocardium can be done very easily.

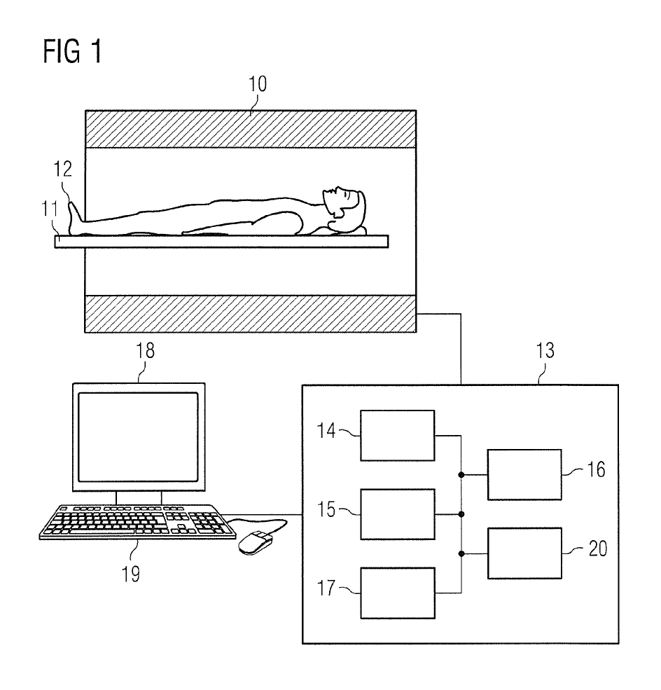

Overall, this means that with the use of the calculated T1 times MR template images can be calculated that have different contrasts, and can be used first for segmenting the myocardium and second for segmenting the scar tissue. FIG. 1 schematically shows an MR system with which determination of the scar tissue fraction in the myocardium is inventively implemented. The magnetic resonance system has a scanner with a magnet 10 for generating a polarization field B0, with an examination person 12 on a couch 11 being moved into the center of the magnet 10 in order to record spatially encoded magnetic resonance signals from an examination region that includes the myocardium. The magnetization of nuclear spins produced by the polarization field B0 can be deflected from the equilibrium state by radiation of radio-frequency pulse sequences and switching operations of magnetic field gradients, and the subsequent relaxation of the nuclear spins from the deflected magnetization results in the emission of magnetic resonance signals that are detected by receiving coils (not shown). The general operation of a scanner for creating MR images and the detection of magnetic resonance signals are known to those skilled in the art, so a more detailed description is not necessary herein.

The magnetic resonance system also has an MR control computer 13 for controlling the MR apparatus. The central MR control computer 13 includes a gradient controller 14 for controlling switching of the magnetic field gradients and an RF controller 15 for controlling and radiating the RF pulses for deflecting the magnetization. The imaging sequences necessary for recording the MR images can be stored in a memory 16 along with all programs that are necessary for operating the MR system. A sequence controller 17 controls image recording and thereby controls the sequence of the magnetic field gradients and RF pulses dependent on the chosen imaging sequences. The sequence controller 17 therefore also controls the gradient controller 14 the RF controller 15. MR images can be reconstructed in an image computer 20, and these images can be displayed on a display monitor 18. An operator operates the MR system via an input interface 19. The image computer 20 is also designed to quantify a scar tissue fraction in the myocardium, as will be explained in detail below.

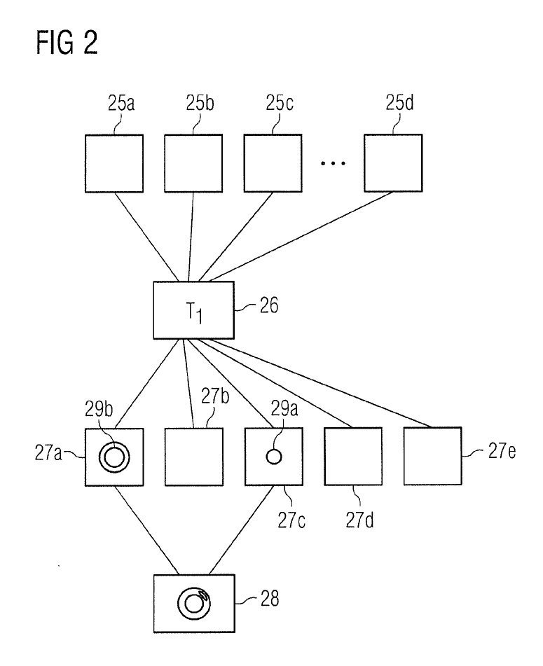

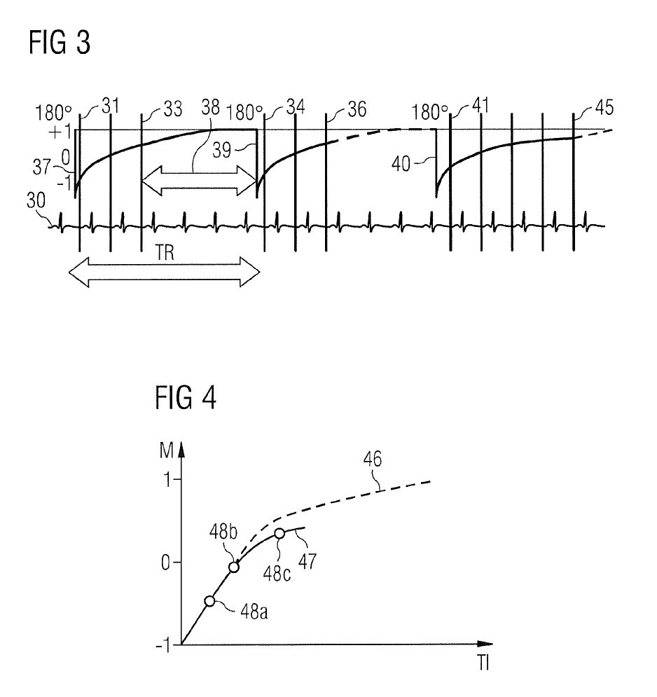

FIG. 2 schematically shows how the different template images can be calculated from the recorded MR data and the generated MR images in order to determine the myocardial volume and scar tissue. A number of MR images is generated for this purpose and these are brought into registration with each other, so MR images 25a-25d result. FIG. 3 shows a possible pulse sequence for generating the MR images 25a to 25d. FIG. 3 shows how the ECG signal 30 of the examination subject is recorded and how different MR images are optimally recorded in the same cardiac phases, with recordings of the individual MR images being shown as vertical lines 31 to 36.

After radiation of an inversion pulse 37, a 180° pulse, the relaxation of the magnetization is detected at different times after inversion, by recording MR raw data for a number of MR images. The images can be recorded, for example, with fast gradient echo sequences. In the illustrated example three MR images 31 to 33 are recorded after radiation of the first inversion pulse 37 with different contrasts as a function of the chosen inversion time. A period 38 then elapses in which the magnetization recovers again before the next inversion pulse 39 is radiated, followed by the recording of three further MR images with the imaging sequences 34 to 36. Three cardiac cycles will have elapsed again before irradiation of the third inversion pulse 40, followed by four further imaging sequences 41-45. In the illustrated exemplary embodiment, images were recorded after inversion pulses 3,3 and 5. Three heartbeats in each case will have elapsed in-between before the fourth heartbeat was taken as the trigger for the next inversion pulse. This can be described as 3(3)3(3)5, with the times in brackets indicating the heartbeats between the inversion pulses.

A further possibility of image recording would be, for example, 3(0)2(0)2(0)1. In this embodiment three MR images would be recorded after the first inversion; no waiting time would elapse for more complete relaxation of the magnetization, instead the next heartbeat would be used as the trigger for the next inversion pulse. Two MR images would then be generated after the next inversion pulse, and thereafter two images after the third inversion pulse and one MR image after the fourth inversion pulse, again without a waiting time.

The inversion time T1 is adjusted here such that good coverage of the anticipated T1 times is ensured. One example would be TI=160,200,240 and 280 ms. In this embodiment the magnetization cannot relax in its state of equilibrium. Since it is not a matter of absolute quantification of the T1 time, however, but merely a matter of a ratio between scar tissue and myocardium size, sufficient relaxation is not so relevant, so time can be saved here when imaging.

This is shown in more detail in FIG. 4, in which the magnetization is shown scaled to the size between 1 and −1 as a function of the inversion time. Directly after radiation of the inversion pulse, a 180° tilting of the magnetization takes place, so the magnetization is at −1 and thereafter relaxes in the direction of the state of equilibrium. Due to the effect of the signal readout with RF pulses and readout gradients, the magnetization does not relax back into the state of equilibrium, as is indicated by curve 46, and instead the magnetization follows curve 47. Furthermore, three scanning points 48a to 48c are indicated as an example in FIG. 4, and these originate from three MR images recorded during relaxation. It is then possible to calculate the T1 time from these three or this plurality of points. Either a two parameter or a three parameter model can be used as the basis here. For a two parameter model, a fit through points 48a to 48c is made according to the following equation:

S=A(1−EXP(−TI/T1))

The equation of magnetization or of the signal is the same in the case of the three parameter model

S=A−B EXP(−TI/T1)

Since, as mentioned, the next inversion pulse can be radiated before the magnetization returns to the state of equilibrium, expanded fitting methods having more parameters can also be used.

Referring to FIG. 2 again, the MR images 25a to 25d would then be generated at the different inversion times, as was described above together with FIGS. 3 and 4, with the residual movement between the images being suppressed by registering. These images all have a different contrast owing to the different inversion time TI. As described in conjunction with FIG. 4, a T1 time can be calculated from the signal characteristics in the individual pixels, so a T1 map 26 can be generated for the examination region comprising the myocardium. When the T1 time is accordingly known, MR template images can be calculated for the different image points with the aid of the calculated T1 times at different times after the irradiation of the inversion pulse. These different template images 27a to 27d each have a different contrast. In one of these images of the heart, in which the myocardium is mapped, a good differentiation of the myocardium outer wall, for example, can be possible, while the myocardium inner wall can best be identified in the same image or a further different MR template image. The scar tissue 29a itself will be best in a calculated template image, for example, in which the healthy myocardium does not supply a signal and therefore the magnetization passes through the zero line while the scar tissue already exhibits a higher signal. FIG. 2 shows this schematically in image 27c, so a scar template image 27c is chosen from the template images, on which the scar tissue 29a can be best identified and segmented. In the illustrated example, image 27a is the myocardium template image in which the myocardium 29b with internal and external contours can best be determined. The contours identified in the template images 27a and 27c can then be combined in a single image 28. This combining is possible since the images 25a to 25d were registered on each other, so residual movements due to respiration were eliminated. In one embodiment the images 25a to 25d were all recorded during the same cardiac phase and in a single breath-holding phase in order to be able to generate the same movement state between the individual images. A residual movement can be compensated by registering of the individual images on each other. Segmenting of the myocardium as in the myocardium template image 27a can be implemented, for example, with a contrast in which the blood itself is shown dark in the myocardium, what is known as Dark Blood Contrast, or at the end of the relaxation curve when magnetization approaches the state of equilibrium. The scar tissue can be determined best, for example, in a template image in which the healthy myocardium does not supply a signal component or supplies a very low signal component.

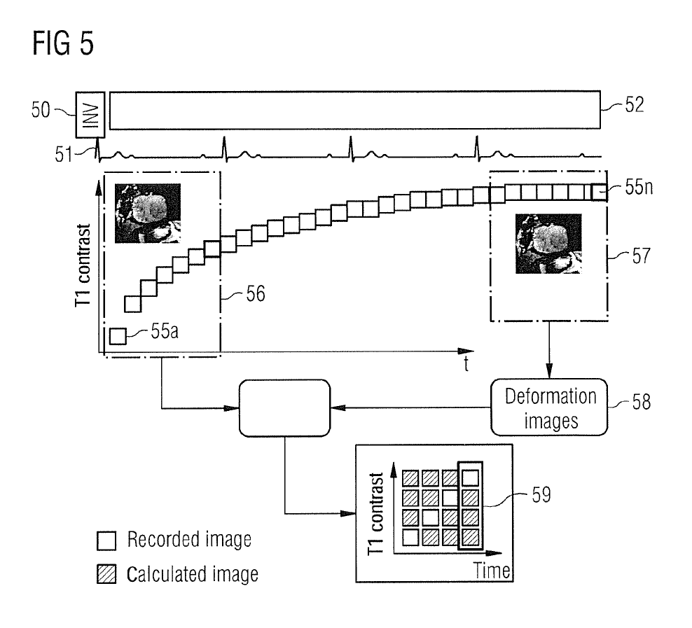

FIG. 5 shows a further embodiment. As described above in the embodiment of FIG. 3, the same cardiac phase was used for recording the MR signals. In a further embodiment it is possible to record image data continuously after irradiation of an inversion pulse. An inversion pulse 50 is initiated by ECG triggering 51. Recording of the MR signals in a plurality of MR images then follows in one period, and this is shown by the bars 52. The imaging sequence used can be, for example, a BSSFP sequence. Many different MR images 55a to 55n are recorded here that belong to different cardiac phases. In the illustrated case recording takes place over four cardiac cycles. The temporal resolution of the MR images recorded during the different cardiac cycles can be between 30 and 40 ms, so a plurality of MR images can be recorded per cardiac cycle. FIG. 2 schematically shows the contrast, moreover, which the individual images have to satisfy. As can be seen, the contrast changes greatly between the individual MR images in the cycle 56 due to the inversion pulse which has just been irradiated. The magnetization approaches its state of equilibrium over the recording time, so in the last cardiac cycle 57 the difference in the magnetization between the individual MR images is only very slight.

The MR images in cycle 57 can then be used to calculate movement information of the moving heart, for example deformation information. Since the MR images have a slight difference in contrast in cycle 57, the cardiac movement can be easily determined using these images since no differences in contrast caused by tissue occur between the individual images. Those skilled in the art know how registering of the individual MR images is possible in the case of different cardiac phases and how individual items of deformation information can be calculated therefrom that show the deformation of the heart in the individual cardiac phases.

It is thereby possible to calculate deformation images as is schematically shown in FIG. 5 by the field 58, so the cardiac movement can be identified which can be applied to the MR images in the first cycle 56. This is described in more detail in German application 10 2014 206724. As is described in detail in this document, it is accordingly possible to calculate images with further T1 contrasts for the different inversion times and cardiac phases. As is indicated by frame 59, one of the cardiac phases can then be chosen for segmenting. Once a cardiac phase has been selected, as symbolized by frame 59, the myocardium can then be segmented in one template respectively, the myocardium template, from the different templates in the frame 59 and the scar tissue can be segmented in a different template. In the embodiment of FIG. 5 it is also possible to optimize scar quantification by calculating a scar volume and a myocardial volume respectively for various cardiac phases. The scar fraction in the myocardial volume can then be averaged over various cardiac phases. The image with the optimized T1 values is preferably chosen automatically for segmenting of the myocardium or for segmenting of the scar tissue.

FIG. 5 combines the steps of the method for scar quantification.

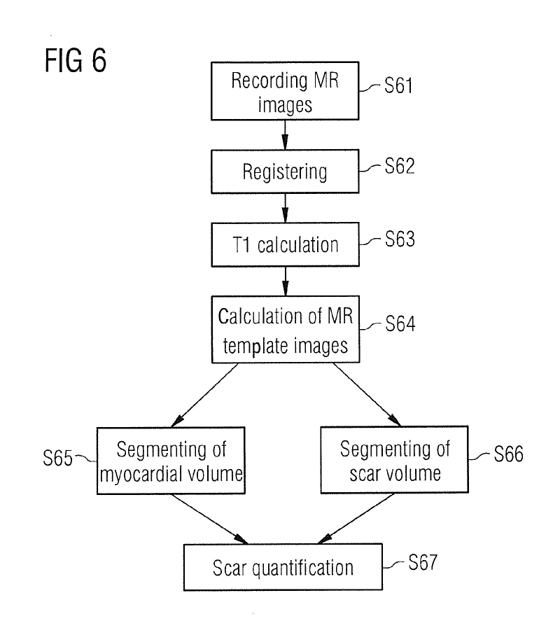

The steps for scar quantification are combined in FIG. 6. The MR images are recorded in a first step S61, wherein, as shown in FIG. 3, recording can occur in a single cardiac phase, or as described in FIG. 5, over various cardiac phases.

Registration then takes place in step S62. With recording of the MR images over a single cardiac phase only residual movements due to the respiratory movement or variabilities in the cardiac frequency have to be compensated here. Translation and rotation can also be taken into account by way of determination of the deformation images with recording over a plurality of cardiac phases. By taking into account the translation and rotation or compression movement different cardiac phases can also be compared with each other in order to calculate the T1 times.

In step S63 the T1 times in the examination region, which comprises the myocardium in particular, are then calculated. Using the calculated T1 values for the individual image points it is possible in step S64 to calculate the MR template images, as are described in FIG. 2 as images 27a to 27e. The template image(s) in which segmenting of the myocardial volume is best possible can then be automatically selected from these template images. The myocardial volume is therefore segmented in step S65 while the scar volume can be segmented on a different template image, the scar template image, in step S66. The two segmented regions can then be displayed combined in a single image as in image 28 in FIG. 2. A quotient for scar quantification can be formed in step S67 from the respective volumes.

In summary, the invention enables robust and automated or highly simplified scar quantification in a short period of time.

Although modifications and changes may be suggested by those skilled in the art, it is the intention of the Applicant to embody within the patent warranted hereon all changes and modifications as reasonably and properly come within the scope of the Applicant's contribution to the art.

1. A method for determining a fraction of scar tissue in the myocardium of an examination subject, comprising:
operating a magnetic resonance data acquisition scanner to acquire magnetic resonance signals while an examination subject is situated therein, to prepare magnetization of nuclear spins in the subject by radiating at least one radio-frequency (RF) preparation pulse into a region of the examination subject that includes the myocardium;
operating the scanner to acquire magnetic resonance signals from the region while the prepared magnetization returns to an equilibrium magnetization;
in a processor, reconstructing a plurality of MR images of the region from the acquired magnetic resonance signals;
in said processor, bringing said plurality of magnetic resonance images into registration with each other so a movement of the heart between a number of said magnetic resonance images is compensated with different contrasts, and thereby obtaining a sequence of compensated magnetic resonance images;
in said processor, determining T1 times in the region using said sequence of compensated magnetic resonance images;
in said processor, calculating different magnetic resonance template images of the region at different times after said radiating of said at least one RF preparation pulse, using the calculated T1 times, said different magnetic resonance template images having different contrasts in said region;
in said processor, determining a myocardial contour of the myocardium using at least one myocardium template image that has a first contrast, selected from said different magnetic resonance template images;
in said processor, determining scar tissue in the myocardium using at least one scar template image, which has a second contrast that differs from said first contrast, also selected from said different MR template images; and
providing an electronic output from said processor representing the determined scar tissue in the myocardium.

2. A method as claimed in claim 1 comprising operating said magnetic resonance data acquisition scanner to radiate a next RF preparation pulse, after said at least one RF preparation pulse, that produces a magnetization of said nuclear spins that is less than 70 percent of an equilibrium magnetization.

3. A method as claimed in claim 1 comprising, in said processor, determining said myocardial contour using two different myocardial template images that respectively have different contrasts, by determining a myocardium outer limit from a first of said two different myocardium template images, and determining a myocardium inner limit from a second of said two different myocardium template images.

4. A method as claimed in claim 1 comprising acquiring said MR signals during a same movement phase of the heart, over a recording period comprising at least six heartbeats.

5. A method as claimed in claim 1 comprising operating said magnetic resonance data acquisition scanner to acquire said MR signals over different movement phases of the heart, during a recording period comprising less than five heartbeats.

6. A method as claimed in claim 5 comprising, in said processor, for each at least some of said different movement phases:
determining T1 times for the respective movement phase using magnetic resonance images acquired during said respective movement phase;
calculating different template images at different times after radiating said RF preparation pulse; and
selecting the magnetic resonance template images from one of said movement phases, and determining a myocardial volume and scar tissue using a myocardium template image and a scar template image each determined from the magnetic resonance template images for said one of said movement phases.

7. A method as claimed in claim 6 comprising, in said processor, determining said myocardial contour and scar tissue separately in each of a plurality of cardiac phases, and determining an averaged myocardial contour and an averaged scar tissue from respective values thereof in said different cardiac phases.

8. A method as claimed in claim 1 comprising, in said processor, calculating a ratio of scar tissue and myocardial volume, using said myocardial contour, and making said ratio available in electronic form from said processor.

9. A magnetic resonance apparatus comprising:
a magnetic resonance data acquisition scanner;
a computer configured to operate said scanner to acquire magnetic resonance signals while an examination subject is situated therein, to prepare magnetization of nuclear spins in the subject by radiating at least one radio-frequency (RF) preparation pulse into a region of the examination subject that includes the myocardium;
said computer being configured to operate said scanner to acquire magnetic resonance signals from the region while the prepared magnetization returns to an equilibrium magnetization;
a processor configured to reconstruct a plurality of MR images of the region from the acquired magnetic resonance signals;
said processor being configured to bring said plurality of magnetic resonance images into registration with each other so a movement of the heart between a number of said magnetic resonance images is compensated with different contrasts, and thereby obtaining a sequence of compensated magnetic resonance images;
said processor being configured to determine T1 times in the region using said sequence of compensated magnetic resonance images;
said processor being configured to calculate different magnetic resonance template images of the region at different times after said radiating of said at least one RF preparation pulse, using the calculated T1 times, said different magnetic resonance template images having different contrasts in said region;
said processor being configured to determine a myocardial contour of the myocardium using at least one myocardium template image that has a first contrast, selected from said different magnetic resonance template images;
said processor being configured to determine scar tissue in the myocardium using at least one scar template image, which has a second contrast that differs from said first contrast, also selected from said different MR template images; and
said processor being configured to provide an electronic output from said processor representing the determined scar tissue in the myocardium.

10. A non-transitory, computer-readable data storage medium encoded with programming instructions, said storage medium being loaded into a computer system of a magnetic resonance apparatus that comprises a magnetic resonance data acquisition scanner, said programming instructions causing said computer system to:
operate a magnetic resonance data acquisition scanner to acquire magnetic resonance signals while an examination subject is situated therein, to prepare magnetization of nuclear spins in the subject by radiating at least one radio-frequency (RF) preparation pulse into a region of the examination subject that includes the myocardium;
operate the scanner to acquire magnetic resonance signals from the region while the prepared magnetization returns to an equilibrium magnetization;
reconstruct a plurality of MR images of the region from the acquired magnetic resonance signals;
bring said plurality of magnetic resonance images into registration with each other so a movement of the heart between a number of said magnetic resonance images is compensated with different contrasts, and thereby obtaining a sequence of compensated magnetic resonance images;
determine T1 times in the region using said sequence of compensated magnetic resonance images;
calculate different magnetic resonance template images of the region at different times after said radiating of said at least one RF preparation pulse, using the calculated T1 times, said different magnetic resonance template images having different contrasts in said region;
determine a myocardial contour of the myocardium using at least one myocardium template image that has a first contrast, selected from said different magnetic resonance template images;
determine scar tissue in the myocardium using at least one scar template image, which has a second contrast that differs from said first contrast, also selected from said different MR template images; and
provide an electronic output from said computer system representing the determined scar tissue in the myocardium.
